(19) United States
(12) Patent Application Publication
(10) Pub. No.: US 2002/0071844 A1
(43) Pub. Date: Jun. 13, 2002

(54) Method of preventing and treating viral diseases

(76) Inventors: Robert J. Tashjian, West Boylston, MA (US); Hui Wang, West Boylston, MA (US)

Correspondence Address: Mirick, O'Connell, DeMallie & Lougee, LLP, 1700 West Park Drive, Westborough, MA, 01581-3941, US

(21) Appl. No.: 09/919,441

(22) Filed: Jul. 31, 2001

Related U.S. Application Data
(60) Provisional application No. 60/221,793, filed on Jul. 31, 2000.

(51) Int. Cl. A61K 39/42; A61K 39/12; A61K 35/78

(52) U.S. Cl. 424/159.1; 424/204.1; 424/725

(57) ABSTRACT

The prevention and treatment of Acquired Immunodeficiency Syndrome (AIDS) in the human, Equine Infectious Anemia (EIA) in the horse, and other viral diseases of man and animal using antiserum of equine origin in addition to combinations of antiviral biologics and drugs.

### CROSS REFERENCE TO RELATED APPLICATION

This application claims priority of Provisional Application 60/221,793, filed on Jul. 31, 2000.

### FIELD OF THE INVENTION

The prevention and treatment of Acquired Immunodeficiency Syndrome (AIDS) in the human, Equine Infectious Anemia (EIA) in the horse, and other viral diseases of man and animal using an antiserum or other products of equine origin in addition to combinations of antiviral biologics and drugs.

### BACKGROUND OF THE INVENTION

The horse can develop a special kind of cellular immunity when stimulated with the modified live virus of equine infectious anemia (EIA). This is unique because it is the only modified live virus vaccine for a retrovirus. A retrovirus in contrast to other types of viruses survives in the body for life. The immunity that this retrovirus EIA vaccine produces is broad and even protects horses against other infectious diseases in China.

This country has placed most emphasis on the molecular biology of HIV intended to produce a safe, effective HIV vaccine and treatment. However, HIV immunology and replication has been unclear after many years of intensive research. Little progress has been made on the prevention and treatment of lentiviruses except the Chinese live attenuated horse EIA vaccine. The factors that produce this immunity for EIA are unclear.

Animals co-existing for long periods of time can develop a special immunity naturally. The manner in which horses can naturally attenuate a retrovirus is still not entirely known.

EIA and HIV are strikingly similar retrovirus lentiviruses. The expression of clinical disease is similar for both, in addition to the molecular biology of the viruses. EIA is the first known retrovirus, having been reported over 100 years ago, in contrast to the origin of HIV being reported in 1983.

Chinese EIA Attenuated Live Vaccines

There are many component parts of the field strain viruses of EIA and AIDS. Many of these components that stimulate antibody response are damaging, and that is a part of the pathogenesis and immunpathology of these two similar diseases. Molecular biologists have tried to determine the damaging antibodies against those that protect and neutralize. It has been a hit and miss project, and furthermore, they are confronted with the problem of different strains, as these viruses change naturally in the field.

The Chinese attenuated the EIA field virus by passing it 120 times through a donkey leukocyte cell culture system. No one but the Chinese know the component ingredients of the cell culture system, and it has never been duplicated. The Chinese hold this key. However, once the vaccine is available outside of China, molecular biologists can determine the factors that are protective from the vaccine.

The cell culture system has attenuated the EIA virus and no one knows the deletions that have occurred from the field strain virus. Molecular biology can compare field strain virus against vaccine.

EIA-HIV Comic Virus

The EIA-HIV comic virus would be created by splicing a piece or pieces of the HIV gene which induces protective immunity, into EIA vaccine or attenuated virus. This is recombinant technology and can take time because of the complex molecular biological laboratory work. The purpose is that this new virus would stimulate protective immunity for HIV in the horse body, so that anti-HIV biological products such as antiserum can be produced from those horses. The same concept can be used for other retroviral and viral diseases by splicing protective components of genes to the EIA vaccine or attenuated virus, stimulating horses to produce protective immunity against those viruses. There are many molecular biology techniques individually and collectively that would need to be used. The worldwide potentials for medicine, science, and health are enormous.

The Mammary Gland and Other Body Fluids

Antiserum is from the blood, but there are other useful body fluids. For the AIDS and EIA diseases, the body fluids of importance are colostrum, milk, blood, and semen. The colostrum is the first milk drank by the newborn, and it is very dense with antibodies. The newborn intestine is able to absorb these large molecular weight antibodies within the first 48 hours of life, and these antibodies provide protection against many diseases. The milk will continue to carry antibodies, but with nowhere the concentration of the colostrum.

In EIA, the mammary gland can serve as a biological filter and the colostrum, and milk usually has only antibodies and no virus. If a newborn nurses with virus-free milk, there is no chance of obtaining disease through nursing, but if the colostrum and milk contains a virus, it is almost a certainty that the newborn will contract the disease. Therefore, for a mother who is positive but with no virus in the mammary gland, the risk to the newborn is only through blood from the mother. Also, in utero transmission is not too frequent. Colostral and milk products could thus be given immediately to the young of horses with EIA, and humans with HIV.

Clinical Symptoms

EIA is the disease. Both EIA and AIDS express themselves in three clinical forms:

Acute—This is the debilitating form of the disease. In the horse it is referred to as acute EIA. In humans, it is referred to as AIDS.

Subacute—This is a form of the disease that can become either acute or chronic. In humans, this is referred to as ARC (AIDS related complex). The subacute form of the disease can transmit just like the acute.

Chronic or asymptomatic—This form of the disease does not show obvious clinical symptoms. In humans, it is referred to as HIV. This is a terrible misrepresentation, because the asymptomatics can transmit, just like the acute.

### SUMMARY OF THE INVENTION

HIV and EIA are very similar on a molecular basis and immunology. The Chinese live attenuated EIA vaccine is successful. Horses over the course of twenty years have developed a unique natural immunity against EIA. There is much precedent to use horse serum for human biologics, especially antiserums. Based on scientific knowledge and information on the Chinese EIA vaccine, and modern molecular biology technology, an EIA HIV comic virus is possible, made by splicing component parts from the HIV genes which can induce protective immunity for HIV into EIA vaccine or attenuated EIA virus. Also drugs can be combined with vaccines and other biologics to have a combination single treatment for both prevention and cure for viral disease.

It is, therefore, an object of the present invention to provide a method for the prevention and treatment of Acquired Immunodeficiency Syndrome (AIDS) in the human, Equine Infectious Anemia (EIA) in the horse, and other viral diseases of man and animal using an antiserum of equine origin in addition to combinations of antiviral biologics and drugs.

It is a further object of the invention to provide a method in which the antiserum can be manufactured simply, and with a highly reliable and consistent results.

With these and other objects in view, as will be apparent to those skilled in the art, the invention resides in the combination of elements set forth in the specification and covered by the claims appended hereto, it being understood that changes in the precise embodiment of the invention herein disclosed may be made within the scope of what is claimed without departing from the spirit of the invention.

The prevention and treatment of Acquired Immunodeficiency Syndrome (AIDS) in the human, Equine Infectious Anemia (EIA) in the horse, and other viral disease of man and animal using an antiserum of equine origin in addition to combinations of antiviral biologics and drugs. One of the products is EIA antiserum by direct vaccination. This antiserum can be used for immediate protection and treatment of horses with EIA and other equine infectious diseases. Antibodies from this antiserum may even help AIDS patients because of the cross-reactivity of EIA and HIV antibodies.

### DESCRIPTION OF THE PREFERRED EMBODIMENT

Several purposes of the invention are the following:

1. The production of equine infectious anemia (EIA) antiviral products from the horse such as antiserum, and protective antibodies from other natural body fluids as colostrum, milk, serum, and saliva and any other related EIA antiviral products developed from a live attenuated virus of EIA and natural immunity.

2. The production of Human Immunodeficiency Virus (HIV) antiserum and other antiviral products of equine origin developed from a HIV-EIA comic virus.

3. A combination of antiviral biologics and drugs including herbal medicines for man and animal into a product for both prevention and treatment.

4. Diagnostic test kits that will distinguish antibodies from infection and passive immunity, for both EIA and AIDS.

Definitional Section:

Live Attenuated Virus or Vaccine—The field strain virus that causes infection has been attenuated by such methods as cell culture or naturally over a period of time in the animal in order to stimulate protective antibody protection, but not persistent infection.

Natural Immunity—A natural immunity of protective antibodies and cellular products that develop over a period of time without vaccination.

Comic Virus—A recombinant new virus developed by splicing together component parts of two similar viruses as the EIA and AIDS viruses. Both EIA and AIDS are similar retroviruses (persist in the body for life) and are sub-classified as lentiviruses because of similar clinical and molecular and biological characteristics. The purpose of this comic virus is to have a virus with EIA and AIDS components that can stimulate protective antibodies after injection into the horse. It will be a species specific virus, and not air-borne, for the horse. It will also contain an antibiotic sensitive gene so that the virus can be destroyed in vitro or in vivo by an antibiotic.

Herbal Medicine—These are medications of botanical and plant origin representing many years of Chinese combinations of these medicines. Certain herbal medications also include those of animal origin.

Antiserum and Passive Immunity—Protective antibodies are produced due to infection of vaccination. However, in lentivirus diseases that persist, the antibody production is not all protective and some of the antibodies will be part of the immunopathology of causing persistent infection and disease. The purpose of the comic virus in this invention is not to have antibodies that cause disease.

### Technology and Methodology

1. Live attenuated EIA virus:

A. Method of attenuation including the Chinese EIA vaccine, EIA attenuated viruses, and naturally attenuated EIA Viruses.

(a) Genetic Engineering Techniques:

The use of molecular biological methods to analyze the Chinese Attenuated EIA Vaccine, clearly understanding the attenuation mechanism of the Chinese EIA Vaccine, according to the information obtained from the Chinese EIA Vaccine. The use of molecular biological methods to create a safe, effective, attenuated EIA virus, that will provide protection and not persistent infection.

(b) Chinese EIA Live Attenuated Vaccine:

The Chinese EIA attenuated Vaccine is attenuated by passing through a donkey leukocyte culture for more than 120 passages. This vaccine can be inoculated into healthy horses by subcutaneous, intravenous injection stimulating the humoral and cellular immunity without any pathogenesis. The serum from inoculated horses does not contain virus and the vaccine replicates partially in the horses. After about 6 months post-inoculation, the vaccine only expresses the envelope (ENV) gene. The Glycoprotein is persistently expressed in vivo from horses and stimulates strong humoral and cellular immunity. This vaccine can be directly used to produce EIA antiserum and other related antiserum and other related anti-EIA products.

B. Characteristics of the live attenuated EIA virus.

(a) Replication: The EIA attenuated virus must be able to replicate in healthy horses by subcutaneous, intramuscular, and intravenous inoculation, and all parenteral routes of administration.

(b) Incomplete replications: After inoculation into the horses, the EIA attenuated virus must not replicate totally and form the whole virus. The blood serum from horses inoculated with this attenuated virus are virus free.

(c) Partial expression: The attenuated EIA virus must express the ENV gene predominantly, so that the ENV gene products remain in the horse for adequate time to stimulate the immune system.

(d) Immunogen: The attenuated virus must have adequate immunogenic features which can stimulate the horse to produce high level humoral and cellular immunity.

2. EIA antiserum and related products:

A. The use of an EIA attenuated virus to inoculate into healthy horses, and establish the basic cellular and humoral immunity against the EIA virus.

B. After 6 months post-inoculation, the levels of immunity are enhanced in these horses with whole cell killed EIA virulent viruses from different EIA strains.

C. The use of Humoral and cellular diagnostic immunity tests to determine the levels of humoral and cellular immunity of horses previously inoculated.

D. The use of blood, other body fluids and organs from horses with strong humoral and cellular immunity to manufacture the EIA antiserum and other anti EIA products.

3. HIV antiserum of equine origin using the live attenuated virus:

A. Construction of an HIV-EIA attenuated comic virus

(a) Construction of an EIA attenuated virus infectious complement DNA (cDNA).

(b) The use of molecular biological techniques to cut the ENV gene from HIV cDNA. The ENV gene must contain the code of the determinates which can stimulate humoral and cellular immunity in vivo.

(c) The use of molecular biological techniques to cut the ENV gene from attenuated virus. The cut cannot affect the EIA attenuated virus inoculation results and the partial replication in the horse.

(d) The use of genetic engineering techniques to insert the ENV gene from HIV into the EIA attenuated virus.

(e) The use of molecular biological techniques to modify the comic virus and make certain this comic virus has the following features:

i. The comic virus must have the ability to replicate in the horse by subcutaneous, intramuscular, and intravenous injection, and also have all the features of the attenuated EIA virus.

ii. The comic virus must predominantly express the ENV gene in the horse.

iii. The comic virus must partially replicate in the horse and not form the whole virus. The comic virus must persistently express HIV ENV gene in the horse for a long period of time.

B. Inoculation of horses with EIA-HIV comic virus

Healthy horses are inoculated with the above EIA-HIV comic virus by subcutaneous, intramuscular, or intravenous routes.

C. Monitor the EIA-HIV comic virus in vivo in horses:

The use of Polymer Chain Reaction (PCR), Probe, and Western Blot techniques to monitor the EIA-HIV comic virus in vivo in horses. Verify the serum of the horses does not contain any virus, and no EIA-HIV comic virus is intact in vivo. Make certain the HIV ENV gene can express in the horses predominantly.

D. Evaluation of the antibodies and cellular immunity levels against HIV stimulated by the EIA-HIV comic virus.

The use of humoral and cellular test techniques to evaluate the levels of humoral and cellular immunity against HIV in horses in vivo.

E. Preparation of anti-HIV products:

The use of blood, other body fluid and organs from horses which have strong humoral and cellular immunity against HIV to manufacture anti-HIV products. Simultaneously, some proteins such as CD4 receptor from human lymphocytes are injected into horses, which will stimulate the horses to produce antibodies which can help or kill or restrict the HIV virus.

4. Antiviral treatment with combination of biologics and drugs including herbal drugs and products.

A. Biologics

(a) Antibodies directed against the specific virus to be treated.

(b) Interferon produced from the species to be treated.

(c) Cellular immunity factors such as interleukins.

(d) Viral proteins from the specific virus being treated.

(e) Viral genes from the specific virus being treated.

B. Chinese Herbal Medicine or Chemical Medicines.

(a) Chinese herbal medicine or chemical medicine with antiviral activity.

(b) Chinese herbal medicine or chemical medicine with immunostimulating activity.

(c) Chinese herbal medicine or chemical medicine with anticancer activity for onogenic cancers.

(d) Chinese herbal medicines or chemical medicine directly focused to specific clinical symptoms of the disease such as anemia, fever, etc.

(e) The above herbal drugs are combined together as Chinese traditional medical method to achieve the results listed in a,b,c, and

d. This can require a combination of over thirty herbal drugs, and must be integrated with the molecular biology component of the total drug.

C. Methodology—using Equine Infectious Anemia (EIA) as an example:

Liposome techniques are used to combine herbal or chemical medicine with viral proteins, genes, interferon and cellular immunity factors. Antivirus antibodies are adhered onto the surface of commercially available liposomes, and the drugs and other biologics are inside the liposomes.

The protocol for the retrovirus lentivirus EIA which is basically the same protocol for the other diseases is as follows:

1. Culture the field strain virus.

2. Purify the virus.

3. Disrupt the virus—with enzyme technology. Specific enzymes and chemical reagents are used to disrupt the virus envelope and virus proteins and genes. The best possible combination of reagents and enzymes must be evaluated.

4. Parallel steps will be occurring after the period the above three steps have been complete, and these are:

a. Interferon preparation—The specific interferon is prepared from the animal species. The disrupted virus with Chinese herbal medicine that specifically induces the body to produce interferon is administered. An EIA negative horse is inoculated one week to ten days apart for a total of four times. The first three subcutaneous or intramuscular injections are with disrupted virus. The fourth injection is intravenous and ten days from the third injection which includes disrupted field virus and the Chinese herbal medicine that induces the interferon. After the fourth injection between twenty-four and forty-eight hours, a blood sample is taken from the horse and the serum isolated and test for interferon levels by standard accepted methods which must be higher than ten units if the serum is to be used. The antibody level is also tested with a minimum requirement if it is to be used.

b. Above interferon also can be bought from commercial products for animals and human beings.

c. Combining the biologics and the herbal medicine

i. Using EDTA, or other liposome technology to combine all of the drugs together. Use an electron microscope to confirm the drugs are properly combined.

ii. Prior to the time the drug is totally combined, toxicity and effectiveness are evaluated in vitro and in vivo with mice, rabbits, and virus culture systems. This is a detailed evaluation program.

d. Treatment

i. The effective treatment of infected patients for evaluation requires at least three months.

ii. For example, for EIA, three criteria are used for effective treatment and these are elimination of antibody, elimination of virus by blood isolation, and use of DNA probe to make certain the proviral DNA has been eliminated in the treated horses.

5. Diagnostic test kits that will distinguish antibody from infection and antiserum or vaccination for both EIA and AIDS.

In summary, the following are the basic components of this invention.

### Live Attenuated Eia Virus

### Eia Antiserum And Related Products

### Hiv Antiserum Of Equine Origin Using The Live Attenuated Virus

### Antiviral Treatment With Combination Of Biologics And Drugs Including Herbal Drugs And Products

### Diagnostic Test Kit To Distinguish Antibody From Infection And Antibody From Passive Immunty For Both Eia And Aids

Applications:

1. EIA antiserum

A. Immediate protection against EIA

B. Treatment of EIA positive horses

2. EIA antibodies from colostrum and milk

A. There is a high concentration of antibody in the colostrum and milk of horses from natural infection, and the same for horses inoculated only with EIA attenuated virus, that is also a source of protective antibody.

3. EIA cellular immunity factors

A. Emulsification from spleen and other organs such as lymph nodes can produce cellular immunity factors that protect against EIA after inoculation with live attenuated vaccine or virus.

4. HIV antiserum

A. Immediate protection against HIV.

B. Treatment of HIV and AIDS patients.

C. Special application for prophylaxis such as the antibody in a gel as part of a condom.

5. HIV cellular immunity factors

A. Emulsification from spleen and other organs such as lymph nodes from the horse can produce cellular immunity factors that protect against HIV and AIDS after inoculation in the horse with the comic virus.

6. Combination drugs

A. Vaccine and drug treatment in combination for this product to both prevent and treat, and selectively inhibit or destroy the virus. The combination of drug treatment, immune system stimulation, and biologic missile technology (to attach to specific target cells and viruses).

7. HIV antibodies from other sources than blood

A. Mammary gland—The colostrum and milk from horses inoculated with the EIA-HIV comic virus can also be a rich source of antibody that can have the same applications as the antiserum.

B. Serum—Antibody is also present in the semen and may in special circumstances be harvested from the horse that has been successfully vaccinated with the EIA-HIV comic virus. This can also be a source of antibody.

Minor changes may be made in the form and construction of the invention without departing from the material spirit thereof. It is not, however, desired to confine the invention to the exact embodiments herein shown and described, but it is desired to included all such as properly come within the claims.

The invention having been thus described, what is claimed is:

1. A method of preventing and treating Acquired Immunodeficiency Syndrome (AIDS) in the human, Equine Infectious Anemia (EIA) in the horse, and other viral diseases of man and animal, using at least an antiserum of equine origin.

2. The method of claim 1 comprising using an HIV-EIA comic virus.

3. The method of claim 1 further comprising the addition of antiviral biologics and drugs.

4. The method of claim 1 wherein the antiserum is produced in horses.

5. The method of claim 4 wherein the horses are inoculated with an EIA attenenuated virus.

6. The method of claim 5 wherein tissue from post-inoculation horses is used to manufacture the antiserum.

7. The method of claim 2 wherein the comic virus comprises replacement of the ENV gene of the attenuated EIA virus with the ENV gene from HIV cDNA.

8. The method of claim 3 wherein the drugs include herbal medicines.
